(19) United States
(12) Patent Application Publication
(10) Pub. No.: US 2017/0369924 A1
(43) Pub. Date: Dec. 28, 2017

(54) METHOD FOR MICROBIAL ENRICHMENT

(71) Applicants: Linda Pilarski, Edmonton (CA); Alexey Atrazhev, Edmonton (CA); Jana Lauzon, Edmonton (CA); Cammika Manage, Edmonton (CA)

(72) Inventors: Linda Pilarski, Edmonton (CA); Alexey Atrazhev, Edmonton (CA); Jana Lauzon, Edmonton (CA); Cammika Manage, Edmonton (CA)

(21) Appl. No.: 15/630,327

(22) Filed: Jun. 22, 2017

Related U.S. Application Data
(60) Provisional application No. 62/353,532, filed on Jun. 22, 2016.

(51) Int. Cl. C12Q 1/24; C12Q 1/68

(52) U.S. Cl. CPC C12Q 1/24; C12Q 1/689; C12Q 1/6806; G01N 2333/245; G01N 2333/255; G01N 2333/195

(57) ABSTRACT

The present invention is directed to methods for the growth, enrichment and isolation of microbes within a sample for further use in presumptive identification of pathogenic bacteria that may therein be contained.

### FIELD OF THE INVENTION

The present invention pertains to culture and enrichment methods useful for screening of samples arising in the food processing industry.

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

The presence of pathogenic bacteria in products intended for human consumption is a significant concern, and the subject of both voluntary and government mandated monitoring, testing and reporting. Companies and individuals producing food and water for human consumption conduct frequent pathogen testing to comply with strict food safety regulations. Unfortunately, traditional methods for performing this testing have not advanced in many decades, and come with significant drawbacks.

Current testing protocols involve culture of material from meat products with serological or molecular confirmation. Since there continue to be recalls of beef after the product has been moved to the retail or food service market and illnesses continue to be reported, the efficacy of current sampling plans remains an issue. Culture methods for recovery of bacteria from meat samples are well-described and verified, but they are time consuming, with significant constraints. Pathogen contamination in meat has a very significant economic impact on the meat industry and a serious health impact for the public.

The meat industry currently uses multiple intervention strategies, rigorous hazard analysis critical control point (HACCP) plans and end-product testing to assure the microbiological safety of its products. Despite these activities and the significant investments in their maintenance and improvement, bacterial pathogens, including pathogenic strains of E. coli, continue to make their way into the finished meat products and cause foodborne illnesses. E coli can be found on carcasses prior to pasteurizing and chilling, but their numbers are extremely low when meat exits the chillers. Diligent attention to verified prerequisite programs for cleaning and effective HACCP plans can ensure that the numbers of E. coli on “primals” and “subprimals”, as known in the art, remain very low. However, meat is re-contaminated during fabrication processes (Youssef M K, et al Food Control 2013, 31:166-171). There are currently many approaches to characterizing pathogenic bacteria, including Shiga Toxigenic E. coli (STEC)/verotoxigenic E.coli (VTEC), using the polymerase chain reaction (PCR) and immunological methods, for example, but in general these methods are technically demanding and expensive. Culture methods characteristically take 18 hours or longer from sampling to identification of a presumptive positive sample, with a further several days required to confirm the positive sample, making inventory control challenging and increasing the risk of large recalls. Innovation in the beef industry has resulted in production of meat upon which numbers of E. coli are exceedingly low (Yang, X. et al J Food Prot 2012, 75:144-149) and it is becoming challenging to recover them from meat without examining large samples. Determination of how to obtain a representative sample is an issue in the industry, particularly with ground beef and trim which is stored in 2000 lb bins. Current sampling plans involve selection of 5 samples from a bin containing a collection of beef from a multiplicity of cows, assuming that those samples will be representative of the entire contents of the bin. Since there continue to be recalls of beef after the product has been moved to the retail or food service market and illnesses continue to be reported, the efficacy of current sampling plans are suspect. Culture methods for recovery of bacteria from meat samples are well-described and verified, but they are time consuming, with significant constraints.

New approaches are needed to assess, in particular, O157:H7 trim testing results for unusual patterns and unusual trends during a given work shift. It is also essential to implement testing throughout processing, particularly of ground beef and patties, and review results for unusual patterns. Finally HACCP plans for monitoring mechanically tenderized beef require efficient testing systems for VTEC/STEC. For non O157:H7 adulterants, the requirement is to identify the presence of stx1 and/or stx2, the presence of eae, and a priority serotype (O26, O103, O111, O121, O45, O145).

The meat industry needs a process that can satisfy regulatory requirements of Canada and the United States and will rapidly identify deviations in processing control and/or product that is contaminated with bacterial pathogens so that it can respond accordingly. Regulators require an isolate for definitive identification to support regulatory interventions as well as further analysis. Culture isolates can be used for e.g. DNA fingerprinting to contribute information to epidemiological investigations. The challenge is to provide a test or series of tests that will; 1) satisfy regulatory requirements for both the U.S.A. and Canada and 2) provide microbiological data that is representative of the entire test unit such as the contents of a combo bin, and 3) provide the industry with tools to rapidly identify product that is contaminated with STEC. The tools must be within the capability of industry to use, must be shown to be accurate, sensitive and as rapid as possible.

The single largest impediment to the efficient and cost-effective testing of food and water samples arises from the large mass and volume of food and water for sampling, as compared to the small volumes used for efficient and cost-effective detection of pathogenic bacteria. For example, the average beef carcass can weigh over 750 pounds, and effective removal and identification of microbes that may be present within the carcass presents a challenge for the beef industry. To address this, the food and water processing industries take representative samples of the products being processed, which is subjected to testing. Yet even those representative samples can weigh as much as 1 pound or more, representing a fluid volume of greater than 300 mL; orders of magnitude greater than the sample volumes used in most diagnostic testing (<1 mL). Further, it is common in the art to blend, or macerate meat samples in a process referred to as “stomaching” to provide further release of microbial contamination present in the meat, though stomacher preparations of meat matrix contain protein (including immunoglobulins), blood, fat and debris, any of which might compromise the PCR.

It is critical that the presence of even one pathogenic bacteria is detected within a representative sample generated by the food processor. Given the volume disparity between the representative sample and the volume used in testing, this means that the pathogenic microbe, as well as any non-pathogenic microbes expected to be present in the representative sample, must be enriched, isolated, or allowed to multiply so as to provide enable detection within the small volumes used in diagnostic testing. The current industry standard involves addition of growth media to the representative sample, followed by incubation of the representative sample for times exceeding 8 hours, allowing the proliferation of microbes within the sample, which would include proliferation of any pathogenic bacteria within the representative sample. Although effective, the need to incubate the test material for greater than 8 hours means that food processors need to hold and/or isolate food products in anticipation of diagnostic results reporting the presence, or absence, of pathogenic bacteria.

Longer incubation times required by industry standard procedures also mean that some strains or species of pathogenic bacteria are overgrown by non-pathogens or more vigorous pathogens, resulting in an inability to detect the less vigorous strains/species. This type of interference is well known in the pathogen detection industry. Longer incubation times, as common in the art, result in increase of the pathogenic bacteria being tested for, but also result overgrowth by other bacteria, confounding the ability to detect pathogens present in the sample using detection methods known in the art (for example, PCR, serological, or culture methods). For example, a known inoculum of Listeria monocytogenes, which is readily overgrown by E. coli and/or Salmonella species, becomes undetectable after 24 hrs of incubation, due to overgrowth by other bacteria.

The art is in need of an enrichment technique that allows for shorter incubation times to limit the overgrowth by other microbes, thereby masking the presence of low numbers of pathogenic bacteria, such as Listeria species.

Further, the art is in need of a novel means to induce proliferation, thereby enriching the microbial populations present in a food sample; so as to interrogate the sample for the presence of pathogenic bacteria.

### SUMMARY OF THE INVENTION

In one aspect, the present invention provides for a method for generating a volume substantially enriched for microbial content comprising;
placing said sample in contact with a first volume of minimal media
incubating said sample and said growth media at a temperature conducive for the expansion and proliferation of said pathogenic bacteria, for a period of time sufficient to allow proliferation of said pathogenic bacteria;
removing a sample of said growth media;
centrifuging said removed volume, generating a pellet and a supernatant, removing and discarding the resulting supernatant;
re-suspending said pellet in 5 ml of a volume of solution containing an effective amount of magnetite generating a re-suspended pellet;
introducing a magnetic field to the re-suspended pellet so as to substantially attract and localize said magnetite to a maxima of said introduced magnetic field within the volume of said re-suspended pellet;
while maintaining said introduced magnetic field, removing a portion of said fluid, said portion being a clarified sample;
centrifuging said clarified sample, generating a second pellet and second supernatant;
discarding the second supernatant and re-suspending the second pellet in a volume less than the sample of said first growth media; generating a volume substantially enriched for microbial content.

In one embodiment, said effective amount of magnetite is 3% magnetite w/v. In another embodiment said solution containing an effective amount of magnetite is water. In yet another embodiment said solution containing an effective amount of magnetite is growth media. In another embodiment said introduced magnetic field is generated by a neodymium magnet. In a further embodiment the temperature conducive for the expansion and proliferation of the pathogenic bacteria being tested for is 42° C. In another embodiment, centrifuging comprises application of a force of 13,000×g for 5 minutes.

In another aspect, the present invention provides for a method for preparing a sample for PCR interrogation for the presence of pathogenic bacteria comprising;
placing said sample in contact with a first volume of minimal media
incubating said sample and said growth media at a temperature conducive for the expansion and proliferation of said pathogenic bacteria, for a period of time sufficient to allow proliferation of said pathogenic bacteria;
removing a sample of said growth media;
centrifuging said removed volume, generating a pellet and a supernatant, removing and discarding the resulting supernatant;
re-suspending said pellet in 5 ml of a volume of solution containing an effective amount of magnetite generating a re-suspended pellet;
introducing a magnetic field to the re-suspended pellet so as to substantially attract and localize said magnetite to a maxima of said introduced magnetic field within the volume of said re-suspended pellet;
while maintaining said introduced magnetic field, removing a portion of said fluid, said portion being a clarified sample;
centrifuging said clarified sample, generating a second pellet and second supernatant;
discarding the second supernatant and re-suspending the second pellet in a volume of lytic solution generating a lytic suspension; and
raising the temperature of said lytic suspension to 55° C. for a period of time sufficient to lyse all, or substantially all, of the microbial cells contained in said lytic suspension, followed by raising the temperature of said lytic suspension to 97° C. for a period of time sufficient to inactivate all or substantially all of the lytic activity of said lytic suspension.

In one embodiment, said effective amount of magnetite is 3% magnetite w/v. In another embodiment said solution containing an effective amount of magnetite is water. In yet another embodiment said solution containing an effective amount of magnetite is growth media. In another embodiment said introduced magnetic field is generated by a neodymium magnet. In a further embodiment the temperature conducive for the expansion and proliferation of the pathogenic bacteria being tested for is 42° C. In another embodiment, said lytic solution comprises lysozyme and ProteinaseK, and wherein said introduced magnetic field is generated by a neodymium magnet. In a further embodiment the temperature conducive for the expansion and proliferation of the pathogenic bacteria being tested for is 42° C. In another embodiment, centrifuging comprises application of a force of 13,000×g for 5 minutes.

In another aspect, the present invention provides for a kit for enriching the microbial content of a sample suspected to contain pathogenic bacteria comprising minimal media, magnetite, and instructions describing the methods described herein.

### DETAILED DESCRIPTION OF THE PRESENT INVENTION

The present invention provides for the unexpected benefit of using suspended particles of Fe₃0₄ (“magnetite”, particle size of less than 5 micrometers diameter) to enable faster enrichment and isolation of microbes present within a food or water sample, in particular samples with large amounts of fat, protein, or other organic materials which serve to confound, trap or otherwise inhibit the detection of microbes within the sample. This is especially relevant in the food processing industries, in particular the meat processing industry.

Although not necessary to practice the present invention, it is hypothesized that the addition of magnetite to an aqueous suspension which contains microbes selectively binds to protein, lipids and dead microbes; with the magnetite and bound material capable of being substantially isolated within the aqueous suspension through application of a localized magnetic field, such as, by way of non-limiting example, bringing a permanent magnet adjacent to the aqueous suspension.

The present invention arises from the novel observation that substantively less incubation time, being the time for microbial growth and population expansion within an aqueous solution capable of supporting, or encouraging, the growth of microbes (“growth media”); is required, as the present method results in the removal of compounds and factors that might otherwise interfere or confound detection of pathogenic bacteria. Of particular benefit is the finding that the present methods are capable of enriching and isolating cold-stresses pathogens, which is particularly relevant in food safety applications.

Therefore, in accordance with the method of the present invention, introduction of a relatively small volume of growth media to a solid sample is possible, with substantially shorter time periods for incubation, allowing for a concentration of the microbial population which can then be interrogated for the presence of pathogenic bacteria or other organisms with such means as known in the art, such as through the polymerase chain reaction (PCR), serological methods such as enzyme linked immune assay (ELISA), immunomagnetic beads, or any interrogation method that preferentially detects the presence of live organisms within an aqueous sample.

When using the methods of the present invention, the presence of microflora does not compromise most subsequent detection strategies, including but not limited to PCR, serological, or culture detection methodologies as known in the art. The resulting enriched material from the methods as described herein can be then used directly in a molecular detection methodology, without any need for further processing, including but not restricted to PCR, RT-PCR, gel-based cassette PCR, Loop Mediated Isothermal Amplification (LAMP), and antibody-based flow cytometric detection of pathogens.

Advantageously, the methods of the present invention do not inhibit molecular detection of pathogens, for example PCR.

The methods of the present invention provide for a low cost and reliable method to reduce the presence of inhibitors (such as fats, or particulate matter) in the complex fluid sample obtained from a food or water sample following incubation. It successfully meets the challenge of substantial removal of potential inhibitors to produce cleaner samples for the end detection methods; and provides a significant benefit in the analysis of the complex samples common in the food or water industries.

It is critical that the presence of even one pathogenic bacteria is detected within a representative sample generated by the food processor. Given the volume disparity between the representative sample and the volume used in testing, this means that the pathogenic microbe as well as any non-pathogenic microbes expected to be present in the representative sample, must be enriched and isolated. The current industry standard involves addition of a large volume of 1:4 ratio (ie. 325 g of sample+975 mL of growth medium) to 1:10 ratio (325 g of sample with 3.25 liters of growth medium) growth media to the representative sample, followed by incubation of the representative sample for times exceeding 15 hours, often as much as 26 hours, allowing the proliferation of microbes within the sample, which would include proliferation of any pathogenic bacteria within the representative sample. Using the methods of the present invention, detection of pathogenic bacteria is possible within as little as 4 hours, for bacteria such as E. coli, times increasing, or decreasing, depending on the replication/growth rate of the bacterial population of interest.

Although effective, the need to incubate food or water samples for at least 10 hours means that food processors need to hold and/or isolate food products in anticipation of diagnostic results reporting the presence, or absence, of pathogenic bacteria. The longer incubation times required by current industry procedures means that some strains or species of pathogenic bacteria are overgrown by non-pathogens, or more vigorous pathogens, inhibiting the ability to detect the less vigorous strains/species. This type of interference is well known in the art. The shorter incubation times of the present invention enables detection of less vigorous strains before they are overgrown. By way of non-limiting example, a known inoculum of Listeria monocytogenes, which is readily overgrown by E. coli and/or Salmonella species, may become undetectable using standard diagnostic methods after 24 hours of incubation, due to overgrowth by other bacteria. The methods of the present invention allow for detection of, for example, 1-3 cfu of Listeria species using a much shorter incubation time. Anaerobic bacteria such as Campylobacter strains may also be detected though growth and replication of the organism is slower and may take up to 24 hours of enrichment.

The methods of the present invention reduce the incubation time normally associated with enriching microbial populations within a food sample, and in accomplishing these and other objects, there has been provided a method for enriching a microbial population present in a food or water sample comprising administering a minimal amount of growth media sufficient to wet the food sample and allow recovery of at least 1 mL after stomaching or massage of the food sample and a short growth period. Although the method is most preferably used to enrich 50 mL of growth medium for every 375 gm of beef trim, as commonly used in industry, only a portion of the medium is needed for the concentration and further processing steps. Thus, smaller amounts of total liquid are contemplated by the present invention, depending on the complexity of the food material being tested.

The novel addition of magnetic particles (magnetite) to an aliquot of the growth media following incubation at temperatures conducive to proliferation of the pathogens being tested for, followed by removal of the magnetite using a magnetic field effectively removes compounds that interfere with subsequent detection methodologies.

After the depletion of inhibitors by the magnetic particles, the pathogen preparation may be treated with lytic enzymes or factors, such as ProteinaseK and lysozyme, to rupture the bacterial membrane and release nucleic acid for detection or detection and amplification by PCR.

The presence of dead bacteria within a food or water sample complicate the testing results by giving a false positive for the presence of live pathogens, using tests based on detection of nucleic acid or antigens, and unexpectedly it was observed that the methods of the present invention result in the removal of up to 37,000 dead bacteria. E. coli can be found on carcasses prior to pasteurizing and chilling, but the number of live pathogens is extremely low when meat exits the chillers. As the decontamination processes kills surface borne pathogens, dead bacteria may still remain on the meat. Advantageously, many dead bacteria, pathogenic or otherwise, are excluded by the methods of the present invention, preventing false positives.

The methods of the present invention have been found suitable for beef trim, ground beef, chicken pieces, processed poultry products, ready to eat meats including lunch meat and hot dogs, dairy products such as yogurt, and water samples from environmental or oil pipelines. After enrichment/concentration as described herein, pathogens spiked onto 375 gm of beef trim mixed with 50 mL of growth media are reliably enriched and subsequently detected by PCR with an inoculum of 1-3 cfu, with the number confirmed by plating. The methods of the present invention efficiently remove inhibitory components and also any remaining fragments of meat which are often fluorescent and interfere with imaging or detecting amplified nucleic acids using methods reliant on fluorescence, as is common in the art. The methods of the present invention do not require any purification of nucleic acid, as intact bacteria may be added to the PCR reaction, avoiding the need for additional equipment. Based on verification of pathogen number by plating and assessing colony forming units, after spiking with 1-3 E. coli, and application of the methods of the present invention, sufficient bacteria were recovered to provide template for PCR. Beef trim spiked with 1-3 cfu, followed by application of the methods of the present invention gave readily detectable amplified nucleic acids following PCR interrogation using primers specific for the bacteria.

Using primers specific to known gene products in the pathogenic bacteria, Shiga toxin (stx1, stx2) and enterohemorrhagic E. coli (eae, O57) spiked onto beef trim were enriched and isolated using the methods of the present invention as evidenced by detection using PCR.

In some cases, only pathogen levels above a defined cutoff limit need to be identified. In these cases, clarification can occur without the need for any enrichment. One example is the identification of heavily contaminated beef carcasses that lead to what are termed “high event days” where equipment and product on those days require recall. Fast screening to detect these carcasses before they enter the processing chain would be beneficial to the art. Implementation of the methods of the present invention absent incubation of the sample in the presence of minimal media and at a temperature conducive for the expansion and proliferation of the pathogenic bacteria; enables detection of at least 7000 cfu in 325 g of ground beef, as the addition of magnetite removes detection inhibitors or materials that confound pathogen detection. Further, the methods of the present invention remove inhibitors and particulates that could compromise PCR, thereby allowing detection of pathogens in complex matrices such as dairy products, such as yogurt, and fruits, vegetables, and legumes, such as bean sprouts, or water samples drawn from environmental sources. The present invention specifically contemplates use of water samples from environmental sources potentially contaminated with hydrocarbons.

Using the methods described herein, 1-3 E. coli (or Salmonella or Listeria, as applicable) were added to a complex food sample. It was observed that after spiking with 1-3 cfu, 4 hours of incubation results in sufficient proliferation of the bacteria so as to reliably collect and concentrate bacteria for subsequent PCR interrogation (using primers specific for the pathogen) and confirmation of the pathogens presence. In all cases, food samples without added pathogen generated negative results upon PCR interrogation and samples spiked with pathogen all scored as positive, giving 100% concordance (no false negatives and no false positives).

The methods of the present invention can be customized for each sample type, as needed, with assembly of kits specifically designed to handle specific types of samples, the kits containing growth media, magnetite, and instructions for the methods described herein. The addition of magnetite to a sample, application of a magnetic field to the solution and subsequent isolation of the fluid from the magnetite provides a significant improvement to the detection of pathogens in complex fluid samples, such as those arising from contact with food (as commonly used in food safety applications) or fluid samples containing hydrocarbon, lipid or biological products.

The methods of the present invention are suitable for processing complex aqueous fluids, for example pipeline fluids that become contaminated with corrosive bacteria, to remove material that could compromise molecular analysis of microflora.

It is observed that the presence of magnetite of up to 20% w/v in growth media during the incubation does not inhibit growth of pathogens or normal flora.

As used herein “enrichment” means the expansion of the bacterial population within the sample.

As used herein “clarification” means the removal of factors which may inhibit later detection, including dead bacteria, using magnetite as described herein.

As used herein, an “effective amount” refers to the amount of magnetite that will substantially remove non-living organic materials such as proteins, fats, connective tissues, or other materials which will confound analysis of the microbial population. The “effective amount” will vary depending on the nature of the food sample being tested, the amount of suspended organic material within any aqueous growth media within it, and other factors determinable by one skilled in the art. An “effective amount” in any individual case may be determined by one of ordinary skill in the art. Ranges of 2% to 6% w/v, more preferably 3% w/v, are appropriate for use with food samples; or 0.5% to 1.5% w/v, more preferably 0.6% w/v for use with environmental water samples.

The magnetic fields contemplated in the present invention are in the range of 0.05-0.2 Tesla, and are achievable by use of small (1.5-3 cm) rare earth magnets, such as neodymium magnets, electromagnets, or larger ceramic magnets with appropriate magnetic field focusing.

### Example 1: Detection of Low-Levels of Cold-Stressed Pathogenic Bacteria

A single colony of bacterial sample (E. coli) was suspended in 10 mL mTSB (Modified Tryptone Soya Broth, OXOID) (or TSB (Tryptic soy broth, Batco)) and test tube incubated for 18 hours at 37° C. The test tube was then transferred to a 14° C. shaking incubator, 200 rpm, for 4 days. The culture was serially diluted in ice cold 0.1% peptone (Batco), and then added to the food samples described below, with live colony counts determined by serial dilution followed by plating on agar plates.

Beef trim samples (15% fat) were prepared as follows: beef trim was cut into smaller pieces and placed in filter bags (Filtra bags, Labplas Inc. Quebec, Canada) with 375 g of beef trim placed in each bag (approximately 30 pieces). For positive controls, E. coli was added to the surface of the meat; and for cold stressed positive controls, E. coli was added to the surface of the meat and the bags were vacuum sealed and placed at 0° C. for up to 30 minutes. At the time of testing, 50 mL of mTSB at 42° C., or at room temperature, was added to each bag, and they were hand massaged for about 10-20 s. Bags were then heated in a warm water bath at 40° C. for 20 min.

Ground beef samples (25% fat) were prepared as follows: 325 g of the ground beef was placed in filter bags. E. coli and 200 mL of mTSB at 42° C. was added to the bag and the contents hand massaged for about 10-20 s. Bags were heated in a warm water bath at 40° C. for 20 min

Bean sprouts were prepared as follows: 25 g of bean sprouts were placed in a filter bag and crushed with a mallet or a rolling pin. E. coli and 10 mL of mTSB at RT was added to the crushed mixture.

Yogurt samples were prepared as follows: 25 g of 1% milk fat yogurt was placed in a filter bag, E. coli and 10 mL of mTSB at RT was added and hand massaged.

Chicken samples were prepared as follows: one chicken drumstick or one thigh was placed in a filter bag. Salmonella, Listeria, or E. coli (prepared as described above) or all three were added to the surface of the meat. 35 mL of TSB was added to each bag.

Expansion of the bacterial population within the sample (“enrichment”) was performed as follows. Filter bags were placed in a rack such that the bags are squeezed resulting in dispersal of the fluid over all, or substantially all of the volume of meat. For samples containing added E. coli, the samples were incubated for 4 to 5 hrs at 42° C. Samples inoculated with E. coli, Salmonella, and Listeria together were incubated for 7 hrs at 37° C. Bean sprouts and yogurt inoculated with E. coli were incubated for 5 hrs at 42° C. After 4 hours, the filter bags were massaged for about 30 s to homogenize the contents and 5 mL of the enriched media was removed to a 5 mL Eppendorf tube.

Removal of factors which may inhibit later detection, including dead bacteria (“clarification”) was performed as follows. The Eppendorf tube with 5 mL media was centrifuged at 3500 rpm for 10 min, the supernatant was discarded, and 1 mL of 3% magnetite in water (w/v) (Iron(II,III) oxide, Sigma Aldrich) added, vortexed to resuspend the pellet, and allowed to stand for at least 2 min. A magnet was placed adjacent to the tube and the clear liquid was transferred to a 1 mL Eppendorf tube. The clear liquid was centrifuged at 12,000 rpm for 5 min, and the supernatant was discarded. The resulting pellet was found to be greatly enriched for the presence of all bacteria, including the test bacteria added to the filter bag.

Additionally, 50-100 μL of lysis buffer, comprised of 1M Tris, and 0.1 mg/mL Lysozyme (Thermo Scientific), was mixed with the pellet to prepare for detection of nucleic acids using, for example, PCR. In such a case, a magnetic field was applied to the resulting suspension (for example, holding the tube to a magnet to remove any leftover magnetite) and the fluid was transferred into a PCR tube containing 5 μL of 10 mg/mL of ProteinaseK. The mix was heated at 55° C. for 15 min and then 97° C. for 4 min.

Detection of the bacteria was performed through PCR on cassettes with hydrated gel capillaries in a prototype instrument described in the art (Manage, D. P. et al, Lab on a Chip, 2013. 13(13): p. 2576). Briefly, it contains a Peltier device for heating and cooling, a laser for fluorescence excitation, and a CCD camera to acquire the images during PCR and MCA. They are controlled by a microprocessor. A laptop computer running a customized Java-based program was used to control the instrument. The DNA amplification was performed with a pre-denaturation step of 95° C. for 3 minutes, then 35 cycles of 95° C. for 15 seconds, 59° C. for 20 seconds, and 72° C. for 20 seconds, followed by a final amplification of 72° C. for 2 minutes. Upon the completion of the PCR, MCA was performed by heating the cassette from 70° C. to 95° C. and the CCD images were taken at 0.2° C. degree interval. They were analyzed to measure the melting temperature for amplicons in each capillary with ImageJ software (National Institutes of Health, U.S.) using the MicroArray Rectangular Plug-in (Dr Robert Dougherty, OptiNav Inc., Redmond, Wash.) to plot the negative derivative of the fluorescence with respect to the temperature in order to determine the melting temperature (Tₘ) of the PCR products.

### Example 2: Testing of Non-Meat Samples

It has been found that the methods of the present invention are effective with environmental water samples, including those containing hydrocarbon contaminants such as from oil and natural gas pipelines.

25 g of yogurt was mixed with 50 mL of TSB and incubated for 5 hours at 42° C. After incubation, some of the light yellow liquid (clear) separated from the solids. 5 mL of clear liquid was taken for clarification. An Eppendorf tube with 5 mL of the clear liquid was centrifuged at 3500 rpm for 10 min, the supernatant was discarded, and 2.5 mL of 4% magnetite in water (w/v) (Iron(II,III) oxide, Sigma Aldrich) was added, vortexed to resuspend the pellet, and allowed to stand for at least 2 minutes. A magnetic field was applied to the side of the tube, and 2 mL of clear liquid was transferred to a 2 mL tube and centrifuged at 12,000 rpm for 5 min. The supernatant was discarded and the pellet was re-suspended in 100 uL of lysis buffer containing 5 μL of 10 mg/mL ProteinaseK and the mix was heated at 55° C. for 15 min and then 97° C. for 4 min. When utilized for PCR, the clarified product was diluted 1:5.

Additionally, 25 g of bean sprouts were placed in a filter bag and 10 mL of mTSB added, the sprouts were crushed with a rolling pin and incubated for 5 hours at 42° C. After the incubation, 5 mL of the growth media was removed and centrifuged at 3500 rpm for 10 min, the supernatant was discarded, and 2.5 mL of 3% magnetite in water (w/v) (Iron(II,III) oxide, Sigma Aldrich) was added, vortexed to resuspend the pellet, and allowed to stand for at least 2 minutes. A magnetic field was applied to the side of the tube, and 2 mL of the resulting clear liquid was transferred to a 2 mL tube and centrifuged at 12,000 rpm for 5 min. The supernatant was discarded and the pellet re-suspended in 100 μL of lysis buffer containing 5 μL of 10 mg/mL ProteinaseK and the mix was heated at 55° C. for 15 min and then 97° C. for 4 min. When utilized for PCR, the clarified product was diluted 1:5.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

What is claimed is:

1. A method for enriching the microbial content of a sample suspected to contain pathogenic bacteria comprising;
placing said sample in contact with a first volume of minimal media
incubating said sample and said growth media at a temperature conducive for the expansion and proliferation of said pathogenic bacteria, for a period of time sufficient to allow proliferation of said pathogenic bacteria;
removing a sample of said growth media;
centrifuging said removed volume, generating a pellet and a supernatant, removing and discarding the resulting supernatant;
re-suspending said pellet in 5 ml of a volume of solution containing an effective amount of magnetite generating a re-suspended pellet;
introducing a magnetic field to the re-suspended pellet so as to substantially attract and localize said magnetite to a maxima of said introduced magnetic field within the volume of said re-suspended pellet;
while maintaining said introduced magnetic field, removing a portion of said fluid, said portion being a clarified sample;
centrifuging said clarified sample, generating a second pellet and second supernatant;
discarding the second supernatant and re-suspending the second pellet in a volume less than the sample of said first growth media;
generating a volume substantially enriched for microbial content.

2. The method of claim 1 wherein said introduced magnetic field is generated by a neodymium magnet.

3. The method of claim 2 wherein said effective amount of magnetite is 3% magnetite w/v in water.

4. The method of claim 3 wherein the temperature conducive for the expansion and proliferation of the pathogenic bacteria being tested for is 42° C.

5. The method of claim 2 wherein said effective amount of magnetite is 3% magnetite w/v in growth media.

6. The method of claim 5 wherein the temperature conducive for the expansion and proliferation of the pathogenic bacteria being tested for is 42° C.

7. A method for preparing a sample for PCR interrogation for the presence of pathogenic bacteria comprising;
placing said sample in contact with a first volume of minimal media
incubating said sample and said growth media at a temperature conducive for the expansion and proliferation of said pathogenic bacteria, for a period of time sufficient to allow proliferation of said pathogenic bacteria;
removing a sample of said growth media;
centrifuging said removed volume, generating a pellet and a supernatant, removing and discarding the resulting supernatant;
re-suspending said pellet in 5 ml of a volume of solution containing an effective amount of magnetite generating a re-suspended pellet;
introducing a magnetic field to the re-suspended pellet so as to substantially attract and localize said magnetite to a maxima of said introduced magnetic field within the volume of said re-suspended pellet;
while maintaining said introduced magnetic field, removing a portion of said fluid, said portion being a clarified sample;
centrifuging said clarified sample, generating a second pellet and second supernatant;
discarding the second supernatant and re-suspending the second pellet in a volume of lytic solution and adding said suspension to a volume of proteinase solution generating a lytic suspension; and
raising the temperature of said lytic suspension to 55° C. for a period of time sufficient to lyse all or substantially all of the microbial cells contained in said lytic suspension, followed by raising the temperature of said lytic suspension to 97° C. for a period of time sufficient to inactivate all or substantially all of the lytic activity of said lytic suspension.

8. The method of claim 7 wherein said introduced magnetic field is generated by a neodymium magnet.

9. The method of claim 8 wherein said effective amount of magnetite is 3% magnetite w/v in water.

10. The method of claim 9 wherein the temperature conducive for the expansion and proliferation of the pathogenic bacteria being tested for is 42° C.

11. The method of claim 7 wherein said effective amount of magnetite is 3% magnetite w/v in growth media.

12. The method of claim 11 wherein the temperature conducive for the expansion and proliferation of the pathogenic bacteria being tested for is 42° C.

13. The method of claim 7 wherein the lytic solution is comprised of lysozyme and ProteinaseK.
